(19) United States
(12) Patent Application Publication
(10) Pub. No.: US 2014/0296948 A1
(43) Pub. Date: Oct. 2, 2014

(54) NEW THERAPY OF CANCER WITH PULSED RADIO FREQUENCY

(71) Applicant: HITOPS GMBH, Kerns (CH)

(72) Inventor: Menno Emanuel Sluijter, Kerns (CH)

(73) Assignee: HITOPS GmbH, Kerns (CH)

(21) Appl. No.: 13/851,427

(22) Filed: Mar. 27, 2013

(51) Int. Cl. A61N 5/00

(52) U.S. Cl. CPC A61N 5/00
USPC 607/101

(57) ABSTRACT

The invention relates to a method for medical treatment of a mammal, preferably a human, suffering from cancer by applying pulsed radiofrequency (PRF) stimulation before chemotherapy or hormonal therapy. The PRF treatment may be given intravascularly or transcutaneously.

In another embodiment, PRF treatment is performed through transcutaneous application of the signal.

### FIELD OF THE INVENTION

The invention relates to the field of medicine, more particularly the field of treatment with electrical signals, more specifically the treatment of cancer and other neoplasias.

### BACKGROUND OF THE INVENTION

Use of electrical current, especially in the area of anodynia, more specifically in the area of pain relief, has been known for several decades. The first reports of the use of electrical current for pain management appeared in the 1930's. Initially, DC was used to induce lesions of nerves through the temperature increase caused by the electrical current (thermocoagulation). Later, this has been replaced by AC with a frequency of 400 to 500,000 Hz, which has been shown to deliver more precise lesions. In the past few decades this radiofrequency (RF) thermocoagulation has been established as an accepted treatment option for trigeminal neuralgia, for unilateral cancer pain and for zygoapophyseal joint pain. Further, RF has been used in other fields:

in cardiology for thermocoagulation of conductive tissue in the heart that conduct aberrant stimulus patterns;

in oncology for destroying tumor tissue;

in orthopedics for treatments of cartilage defects in osteoarthritis.

The therapeutic effect was mainly concerned with the destruction of tissue by the heat that was generated by the current. The development of a novel method for administrating high frequency current, pulsed radiofrequency (PRF) allowed using it to treat other pathologies and nerve structures. With PRF, current is delivered in pulses of short duration (1-100 msec) separated by a silent period of about 0,1 to 1 sec. Output current may be set not to exceed 42° C. to prevent cell destruction. Heat generated by the application of the current is dissipated between pulses. Nowadays PRF is recognised as treatment for e.g. various forms of spinal and facial pain and peripheral neuralgias.

The mechanism through which PRF causes long-lasting pain relief when applied in the vicinity of a nerve is not known.

In the meantime, it has been published by the present inventors (WO 2008/094042) that PRF has beneficial effects on seeds and the germination of seeds. Although also the mechanism that causes these effects is not yet elucidated, it appears that PRF is capable of influencing biological tissue to perform better.

Further, recently it has been published by the present inventor that intravascular application of PRF has several therapeutic benefits next to pain relief, such as boosting the immune system. It was also found to be applicable to treat a disease or condition caused or accompanied by immunodeficiency, such as cancer, infectious diseases, immunosuppression or otherwise caused immunodeficiencies. Also auto-immune diseases and conditions associated with allostatic load have been found amenable for treatment with the method of intravascular PRF.

### SUMMARY OF THE INVENTION

It has now been found that the combination of PRF for the treatment of cancer with use of chemotherapy has surprising effects.

Therefore, the present invention comprises a method for the treatment of patients suffering from cancer or other malignant neoplasms by applying PRF to these patients after which normal cancer therapy, such as chemotherapy or radiation is provided. Preferably, the PRF treatment is given intravascularly. Alternatively, PRF can be applied transcutaneously by using an external plate electrode which is positioned e.g. over the subclavian vessels.

Preferably, the PRF signal is an irregular PRF signal.

In a preferred embodiment, the disease or condition to be treated is caused or accompanied by immunodeficiency, more preferably the disease or condition is selected from the group of cancer, infectious diseases, immunosuppression or otherwise caused immunodeficiencies. Also auto-immune diseases and conditions associated with allostatic load are preferred for treatment with the method of the invention.

In an also preferred embodiment, the PRF stimulation is applied together with vaccination. Also part of the invention is the use of intravascular PRF or PRF, which is applied intravascularly, in therapy, preferably in the therapy of immunodeficiency, more particular in the therapy of cancer, and in the therapy of auto-immune diseases, such as rheumatoid arthritis, COPD, multiple sclerosis, Crohn's disease, and the therapy of conditions associated with allostatic load, such as diabetes and pathologies caused by diabetes, post-traumatic conditions, fibromyalgia, chronic fatigue syndrome, burnout, migraine, depression, dementia, osteoporosis and osteoarthritis.

### DETAILED DESCRIPTION

PRF (pulsed radiofrequency) is a clinically proven method to alleviate pain in cases where pain sensation is due to or transported via peripheral nerves (such as in case of pain caused by pinching a nerve by a slipped disc of the spinal column, facial pain, trauma, etc.). As discussed above, PRF, just like RF, works through applying an electrical AC current to the vicinity of a nerve. Usually a frequency of 400.000-500.000 Hz is used, but the range may vary from 50.000 to 1.000.000 Hz. In PRF, in contrast to continuous RF, the heat that is generated at the tip of the electrode during the active phase of the duty cycle is dissipated during the resting phase of zero, or of appreciably lower voltage. The settings of the current generator should be adjusted so that the mean temperature around the electrode tip does not rise to cell destructive levels, which start from 45° C. upwards. It is allowable that the temperature may briefly rise above 45° C. during the active phase of the duty cycle (the so-called heat spikes), although the biological effects of these ultrashort rises in temperature are not known. However, the spread of heat into the tissues during a heat spike has been predicted to be minimal (<0.2 mm), thereby outruling that a thermal (coagulation or neurotomy) effect is the cause of the clinical efficacy of PRF nerve treatment—at the same time also casting doubts whether the thermal effect would be crucial for RF. The mode of action of PRF has so far not been elucidated. Three possibilities have been suggested. In chronological sequence these are the following:
1. PRF causes modulation of afferent signals in the dorsal horn of the spinal cord;
2. PRF causes ablation by strong electric fields around the needle tip, and possibly by temperature effects during the active phase of the duty cycle;
3. PRF causes a brief expression of pro-inflammatory cytokines, eventually triggering an anti-inflammatory effect by a change of phenotype of immune cells, such as T-lymphocytes.

Ad 1. PRF is in fact a new entity. It does not cause ablation, like in continuous RF, and it also is not considered to be stimulation like in TENS (transcutaneous electrical neurostimulation) or in epidural stimulation. The pulse generally is 420 KHz and this is far higher than the physiological range. This explains why PRF does not cause any sensation to the patient, even at considerable voltages. We may therefore assume that the first synapse in the afferent chain does not depolarize. That does not mean that PRF has no effect on the nervous system. Laboratory experiments have shown that applying PRF to a segmental nerve root is followed by expression of c-fos in the dorsal horn of the spinal cord (Higuchi, Y. et al., 2002, Neurosurgery 850-856; Van Zundert, J. et al., 2005, Anestesiol. 102:125-131). This phenomenon has been named “transsynaptal induction”. It is a sign that PRF causes presynaptal conditioning.

The importance of this transsynaptal activity for the clinical effect of PRF is still a subject of discussion. There could be Long Term Depression (LTD) of higher afferent synapses, but the clinical course following PRF procedures does not seem to be synchronous with an LTD effect. Also, PRF may have an anti-inflammatory effect when an electrode is positioned intra-articularly, in a position that is at considerable distance from any afferent nerve (Sluijter, M. E. et al., 2008, Pain Pract. 8:57-61; WO 2008/069647). This would suggest that there are two parallel effects that may operate either independently or in concert. Any activity inside the central nervous system cannot be ignored however, and it is reasonable to assume that presynaptic conditioning plays at least an additional role in the mode of action.

Ad 2. Although the overall temperature changes in tissue during PRF application are minimal because of the relatively large pulse intervals, the electric fields close to the needle/electrode are very high during the active pulse. Because the high power deposition during the pulse causes a “heat spike”, thermal effects could contribute to destruction too. It has been suggested that these effects could cause a “mini-ablation”, a watered down version of what happens during continuous RF (Cosman, E. R., 2005, Pain Med. 6:405-424; Erdine, S. et al., 2009, Pain Pract. 9:407-417). There are strong arguments contradicting this theory. Both the electric fields and the temperatures around the needle tip fall off very rapidly (<0.2 mm) away from the tip. Tissue destruction during PRF is therefore limited to minuscule dimensions (Cahana, A. et al., 2003, Pain 4:197-202). Also ablation could never explain the beneficial effects of intra-articular PRF procedures.

Ad 3. There are many anecdotal observations of a reduction of the serum CRP level following PRF procedures, concordant with the theory that anti-inflammation plays a role in the mode of action. Possibly the triggering event of this effect is expression of pro-inflammatory cytokines (Van Duijn, B., 2011, “Exploration of anti-inflammatory effect of PRF at the cellular level”, Int. Symp. “Invasive Procedures in Motion”, Nottwil, Switzerland, January 21-22) by the shaking effect of the alternating electric fields on charged molecules in the cell membrane. In the meantime, it has been published by the present inventors that PRF has beneficial effects on seeds and the germination of seeds (WO 2008/094042) and can stimulate growth and differentiation in in vitro tissue cultures (WO 2010/016765). Although also the mechanism that causes these effects is not yet elucidated or follows clearly from the above hypotheses, it appears that PRF is capable of influencing biological tissue to perform better.

Very recently (WO 2011/078676), the present inventors have found that PRF when applied intravascularly, has very surprising therapeutic effects on a variety of diseases or conditions, amongst which cancer, infectious diseases, immunosuppression or otherwise caused immunodeficiencies. Although no theoretical explanation of the observed effects can yet be given, it is assumed that the electrical current that is delivered through the electrode in a blood vessel is transported through the vessel. The electrical resistivity of the vessel wall is 200 times as large as that of the blood, which means that the blood vessel can be regarded as an insulated electrical cable. Although the electrical resistivity of the blood wall vessel will vary according to the type and diameter of the blood vessel, in general venous blood vessel walls will have a resistivity of about 20 to 1000 Ωcm², where the lower values are found in blood vessels where much transport over the vessel wall is taking place (e.g. in muscle veins, Olesen & Crone, 1984, Biophys. J. 42:31-41) and the higher values occur in vessels of the blood brain barrier (Butt et al., 1990, J. Physiol. 429:47-62).

One of the theories is that the effect of the intravascular PRF treatment is a general boost of the (cells of the) immune system. A similar effect has been found in plant seeds (see WO 2008/094042) and tissue culture (see PCT/NL2009/050483), where upon PRF stimulation the vigour of the treated cells was increased, and in intra-articular PRF (see WO 2008/069647) where the pain effective treatment would also involve lymphoid cells. Apparently, the PRF treatment when applied in the blood vessels is thought to be able to stimulate the blood lymphocytes and make them more active. Another possible explanation is derived from U.S. Pat. No. 6,038,478, in which it is disclosed that electrical stimulation is able to attract lymphocytes. However, in this document frequencies of less than 1000 Hz were used and stimulation was only applied to brain tissue.

Further, the use of electrical stimulation to promote bone and soft tissue healing is known (see e.g., J. Black, Clin. Plast. Surg Apr. 12, 1885 (2):243-57).

In another theory, the intravascular PRF treatment is thought to affect the nervus vagus. The vagus nerve (cranial nerve X) has recently been identified as a pathway that impacts on inflammation. The key endogenous mediator of this so-called cholinergic anti-inflammatory pathway is acetylcholine, the principal neurotransmitter of the vagus nerve, which specifically interacts with α7 cholinergic receptors expressed by macrophages and other cell types to inhibit tumor necrosis factor (TNF) production (Tracey K J. The inflammatory reflex. Nature 2002; 420:853-9.). Stimulation of the vagus nerve has been suggested by Giebelen (Thesis, University of Amsterdam, 2008) to be effective in the treatment of inflammatory, more especially, TNF-α mediated diseases, such as Crohn's disease, rheumatoid arthritis and sepsis. However, in this last study the nervus vagus was electrically stimulated by direct application of electrical current with a very low frequency (1 Hz) on the nerve. One additional argument for this last theory resulted from our experiments. In one of the patients it was impossible to use the vena brachialis for introduction of the electrode. Instead, one of the veins on the back of the hand was used. Although the actual administration of the electrical field through PRF was essentially similar, no results of the treatment were found. A possible explanation in view of the vagus nerve theory can be that the distance to this nerve was made larger by using the hand veins in stead of the vena brachialis, resulting in less electrical field to arrive at the vagus nerve and/or nucleus tractus solitarius.

In the course of the experimentation it was also experienced that in some cases it was difficult or impossible to apply intravascular PRF, e.g. because the veins were badly accessible. In those cases, the PRF has been applied transcutaneously by using plate electrodes of 5×5 cm surface. Small plates were intentionally used to optimize the current density. It has been found that this works optimally when one plate electrode is positioned to cover the subclavian vessel(s), while the other electrode was positioned onto the region of the nervus vagus. However, also other positions for the plate electrodes may be chosen such as placement in the liver region in patients with liver metastases and optimalisation of the stimulation circumstances can be easily accomplished by the skilled person.

It has now been found that a combination of PRF and other therapy is very advantageous to treat cancer, especially cancers in late or very late stadia.

Most surprisingly, it has now been found that PRF is especially effective if it is applied before other anti-cancer therapy that may potentially depress the immune system such as chemotherapy or radiotherapy has been started. As will be clear from the experimental part, of a total of 41 patients were treated intravascularly with PRF. Of the 6 patients that received their first PRF treatment before commencement of chemotherapy, all 6 patients are doing well and show remission of the tumors, while from the 35 patients that were treated with intravascular PRF while already receiving chemotherapy 30 died and 5 are still living but exhibiting tumor growth. While these figures, especially those of the second group may seem dissuasive for applying any form of therapy, it should be considered that all of the patients were suffering from stage IV cancer which means a very advanced tumor growth and very often accompanied by metastases. Accordingly, this is the last stadium before death and treatment often is only palliative. Seen in this light it is even more remarkable that PRF treatment prior to any other treatment causes remission or even cure.

The amount of PRF treatments and whether these follow-up treatments are performed when chemotherapy has already started does not seem to be of influence, but this may be due to the low number of treated patients so far. One patient (patient #1) has been cured from an advanced lung carcinoma with only one PRF treatment, another patient (patient #2) suffering from cervix carcinoma received 8 PRF treatments and is now free of any tumors.

In the treatment of cancers, the diseases may be solid cancers and lymphoma's, leukemia or myeloma's. As can be seen from the examples, all types of cancer, including their metastases, are susceptible to the treatment. Although it has ben established in the present invention that PRF is working best when given prior to other therapies in patients that are suffering from a type IV cancer disease, for those patients that have an earlier form of cancer PRF treatment may be an excellent additional treatment next to chemotherapy or radiotherapy, since it ensures a stimulation of the immune system, which otherwise would be impaired by the chemical or radiation treatment.

As has been shown in the present invention, the PRF treatment may be continued in combination with the chemical or other treatment that the patient otherwise also would have received, like medication, radiation treatment, etc.

The instruments for applying PRF to a patient generally comprise a needle-like electrode, connected to a PRF current source and a means for providing connection to earth. The PRF current source (or lesion-generator) will provide, next to the source for the current, also a stimulator function, to check for the proper positioning of the electrode, and the facility of measuring the impedance of the circuit between patient, earth and apparatus. These devices are commercially available (e.g. the NT 2000 Radiofrequency Lesion Generator from Neurotherm, USA) The procedure to apply intravascular PRF is easy to perform and does not need special skills. For intravascular PRF an insulated needle with an exposed tip of minimally 5 mm, but preferably 15-20 mm, is inserted into a blood vessel, preferably a vein. With such a needle the impedance of the system should be below 1000Ω, preferably about 100-600Ω, more preferably about 200-250Ω. Any blood vessel of sufficient size can be used, but preferred are venous blood vessels that are easily accessible, such as the blood vessel in the elbow (vena brachialis), in the thigh (vena saphena) or neck (vena jugularis). Preferably, in view of the role of the nervus vagus in the inflammatory reflex, a vein should be used which provides a minimal distance to the nervus vagus, such as the vena brachialis, the vena brachiocephalica or the vena jugularis externa.

When the electrode is in the proper position and connected to the PRF current source, the patient will be connected to earth (e.g. by a so-called earth-plate) to establish an electrical circuit. Exposure to PRF is then applied. Usual values are a pulse duration of 10 msec and a pulse frequency of 2-5/sec; a voltage of 20-80 V depending on the impedance of the system; and a total duration of treatment of 10-30 minutes. There is however a wide variation in parameters that may be used:

Frequency: 50.000-1.000.000 Hz, preferably 150.000-500.000 Hz

Pulse duration: 0.1-100 msec, preferably 5-20 msec.

Pulse frequency: 1-20/sec, preferably 2-5/sec

Voltage: 10-80 V, preferably 40-60 V

Treatment time: 2-30 minutes

Further the duty cycle may be irregular, with varying pulse duration and pulse frequency, and the voltage may not be brought back to zero during the rest phase.

The PRF treatment is painless (except for the initial insertion of the needle electrode) and no adverse reactions have thus far been observed. In otherwise healthy persons no apparent changes take place.

In stead of intravascular PRF the PRF stimulus may also be provided transcutaneously by using a plate electrode that will be positioned in the neighbourhood of a blood vessel, preferably a large superficial blood vessel such as the jugular vein or artery, or the subclavian blood vessels. Alternatively, a plate electrode may be used on the femoral veins. The second plate (functioning as ‘earth’-plate may be contacted to any body part, but preferably a location in the neighbourhood of the nervus vagus is chosen. The size of the plate electrode and the depth of the blood vessel under the skin will be critical and will be factors in determining the parameter settings of the stimulation, such as voltage and treatment time. In general it should be considered that the voltage should preferably be at least 5 V per cm distance between the electrodes.

For providing an irregular PRF signal, which is one of the preferred embodiments of the present invention, the PRF signals may be generated by RF lesion generators that are commercially available (such as NT 1100 or the NT 2000 RF generators from Neurotherm, Wilminigton, Mass., USA). These only need to be adapted for the present invention by inserting a module that facilitates the development of an irregular signal. An irregular signal according to the present invention is defined as a PRF signal wherein the pulses are fired according to a Poisson distribution or a combination of Poisson distributions. Such an irregular PRF signal has been described extensively in the co-pending application EP 12194957.2. The definition of irregular PRF signal and how to construct such a signal is explained in much more detail in said application and these parts are herein incorporated by reference.

For intravascular placement of the electrode, the electrodes which are currently used for (P)RF are unsuitable since they have a very sharp tip, which could damage the blood vessel wall. Further, because of the nature of the blood vessel, the use of rigid instruments is limited. For this purpose, a suitable intravascular electrode comprises a hollow outer (needle) part, optionally provided with a releasable inner stylet and an inner, true electrode part (or coil), see FIG. 1 of WO 2011/078676. The outer part is completely insulated, and the insulation material may be any non-conductive material which does not react with biological tissue, such as plastics like polyethylene, polypropylene and the like. For ease of penetration through the skin, the underneath tissues and the wall of the vein, it has a very sharp tip. If present, also the stylet, which fits into the hollow needle, may be as sharp-edged as the needle to construe a continuous sharp-edged tip. After perforation of the blood vessel wall, the stylet is removed. The inner part, the actual electrode, is then inserted into the needle and will extend from the hollow outer needle into the blood vessel. The electrode has a blunt end of more than 5 mm, preferably 15-25 mm, and consists mainly of conductive material, such as metal or doped conductive polymers. Preferably, the electrode is flexible to allow it to follow the natural curves and movements of the blood vessel.

Another option is insulation of the tip of the electrode. It is around the tip of the electrode that the electric and thermal fields are strongest, and in tissue applications of PRF electrodes it has been shown that these strong fields may cause a microscopically small area of necrosis around a sharp needle tip. This effect is already minimalised by the fact that in this case the electrode is blunt and residing in the blood stream, but if the tip will often lie very close to the blood vessel wall it is worth taking precaution. By insulating the tip any damage would be prevented or at least further reduced, without affecting the therapeutic effect of applying the PRF current.

In a further embodiment of the present invention, the electrode is an electro-catheter. Such a catheter can advantageously be used to enable placement of the active part of the electrode closer to a target. Such a target can be any structure in the body that qualifies for treatment of anatomically limited pathology, such as for example Crohn's disease and malignant tumors, or an anatomically limited target, such as for example structures in the central or peripheral nervous system. As discussed above, according to one of the theories underlying the efficacy of intravascular PRF, the electrical field should stimulate the nervus vagus. Placing the electro-catheter in a vessel within close proximity of this nerve would thus enable a more direct stimulation of the nerve and hence a more effective treatment. Some of the blood vessels that run quite close to the nervus vagus are the vena jugularis interna and the left or right innominate vein (brachiocephalic vein), and these veins thus seem an optimal place to position the electro-catheter.

The electro-catheter can be a conventional catheter, but it can also have a steerable tip, such as the catheter disclosed in WO 2010/113072. The catheter disclosed in this patent document would ideally be suited for the presently claimed method.

The RF Lesion Generators that are commercially available are suitable for performing this procedure. However, modifications would greatly facilitate this particular procedure. Particularly important is to ensure that the impedance of the system does not fall below 100Ω, because then the danger would exist that with voltages of 60V gas bubbles would be formed in the blood, which of course can lead to (life-threatening) emboli. Systems to limit the impedance and/or current output are known in the electro technical arts and can easily be applied to the commercially available radiofrequency current generators.

### EXAMPLE 1

The following patients that were diagnosed with cancer as indicated were treated with PRF. As standard for intravascular PRF a stimulation of 20 minutes (from January to September 2012 reduced to 15 minutes) with a voltage of 60V and 4 times per second a stimulation of 10 msec of radiofrequency was given. In the treatments from 1 Sep. 2012, an irregular signal (Poisson distribution) was provided with the same average parameters. Following PRF (chemo)therapy was administered as detailed below.
Patient #1 M, age 59
   Diagnosis: Grade 4 nsc lung carcinoma. Adrenal metastasis
   PRF: Apr. 11, 2009
   Chemo: May 8, 2009 until 22 Jun. 2009
   Adrenalectomy: July 2009
      Chemo+Radiotherapy form Aug. 26, 2009 until Oct. 9, 2009
   Outcome: Tumor free up to date without further treatment
Patient #2 F, age 23
   Diagnosis: Wertheim operation for cervix carcinoma at age 21
      Extensive lung metastasis diagnosed in 2010
   PRF: Nov. 3, 2010
      Feb. 7, 2011
      Apr. 8, 2011
      May 5, 2011
      Jun. 21, 2011
      Aug. 4, 2011
      Oct. 17, 2011
      Apr. 23, 2012
   Chemo: Nov. 4, 2010 until Feb. 28, 2011
   Outcome: Tumor free up to date without further treatment
Patient #3 F, age 42
   Diagnosis: mamma carcinoma+3 liver metastases
   PRF: Apr. 22, 2012
      Jul. 1, 2012
      Sep. 2, 2012
      Dec. 11, 2012
   Treatment: Hormonal therapy
   Outcome: 2 liver metastases completely disappeared, 50% remission of the third one
      Excellent general condition
Patient #4 M, age 77
   Diagnosis: Grade 3B nsc lung carcinoma (unresectable)
   First PRF: Mar. 16, 2012
   Chemo: Mar. 22, 2012 until 6 Jul. 2012
   Radiotherapy Aug. 28, 2012 until Oct. 11, 2012
   Outcome: Excellent general condition
Patient #5 M, age 64
   Diagnosis: Aggressive Prostate carcinoma
      Widespread and intensive lymphatic and bone metastasis
   PRF: Dec. 27, 2012
   Treatment: Hormonal therapy
   Outcome: Spectacular improvement general condition
      80% reduction lymphadenopathy (scan Feb. 21, 2013)
Patient #6 F age 67 Uterus carcinoma
   Uterus removed, but lymph gland metastasis not operable
   PRF Nov. 11, 2011
   Treatment: None (refused chemotherapy)
   Outcome: 50% reduction of metastases (June 2012)
      Excellent general condition
         Other stage IV terminal cancer patients that had received treatment before start of the PRF stimulations did not show these spectacular improvements, and although there may be small effects on the growth of the tumour or the metastases, PRF treatment could not prevent worsening of the disease and/or death of the patient. Of the 35 patients 30 are known to have died, the others are still alive but with no amelioration of their condition. The ages and carcinoma types of these 35 other patients matched the ages and carcinoma types of the 6 patients that showed improvements.

1. A method for medical treatment of a mammal suffering from cancer, the method comprising applying pulsed radiofrequency (PRF) stimulation to the mammal before administering chemotherapy or hormonal therapy to the mammal.

2. The method according to claim 1, wherein said cancer is a stage IV carcinoma.

3. The method according to claim 1, wherein the PRF is applied intravascularly.

4. The method according to claim 1, wherein the PRF is applied transcutaneously.

5. The method according to claim 1, wherein the PRF has an irregular duty cycle.

6. A method for applying pulsed radiofrequency (PRF) to a patient wherein said PRF is applied transcutaneously.

7. The method according to claim 6, further comprising positioning a plate electrode over a subclavian vessel.

8. The method according to claim 6 wherein the PRF is an electrical signal consisting of current pulses in a radiofrequency range with a voltage of 10 V to 80 V to be delivered in pulse bursts with a duration of 0.1 msec to 100 msec and burst frequency of 1/sec-20/sec to be delivered by an electrode with an impedance of less than 1000Ω for medical treatment.

9. The method according to claim 1 where the active electrode is an electro-catheter to enable placement of the active part of the electrode closer to the target.

10. The method of claim 1 where the mammal is a human.

11. The method of claim 9 where the electro-catheter has a steerable tip.
